(19) United States
(12) Patent Application Publication
(10) Pub. No.: US 2023/0213537 A1
(43) Pub. Date: Jul. 6, 2023

(54) DETERMINATION OF OXIDIZING SUBSTANCES USING PEPTIDE DEGRADATION

(71) Applicants: Baxter International, Inc., Deerfield, IL (US); Baxter Healthcare SA, Glattpark (Opfikon) (CH)

(72) Inventors: Robert W. Garber, II, Deerfield, IL (US); Kayce Olbrich, Deerfiled, IL (US)

(21) Appl. No.: 18/069,198

(22) Filed: Dec. 20, 2022

Related U.S. Application Data
(60) Provisional application No. 63/295,705, filed on Dec. 31, 2021.

(51) Int. Cl. G01N 33/74; G01N 30/88

(52) U.S. Cl. CPC G01N 33/74; G01N 30/88; G01N 2030/027; G01N 2030/8831

(57) ABSTRACT

An analytical method for detecting presence of oxidizing substances by measuring degradation of peptides may include preparing a test preparation that includes a peptide and a sample. The peptide degrades in the presence of oxidizing substances. The method may include detecting impurities of the peptide in the test preparation in which detected impurities indicate presence of oxidizing substances in the sample. Examples include using high performance liquid chromatography to detect, identify, and quantify impurities of the peptide indicating presence of oxidizing substances in the sample. Exemplary oxidizing substances include peroxide-containing, chlorine-containing compounds, and bromine-containing compounds. Exemplary peptides include vasopressin.

### RELATED APPLICATIONS

This application claims priority to U.S. Provisional Application No. 63/295,705, filed on Dec. 31, 2021. The contents of the foregoing application are hereby incorporated by reference herein.

### TECHNICAL FIELD

The present application relates to the determination of oxidizing substances using degradation of peptides.

### BACKGROUND

Oxidizing substances are used to clean, decontaminate, and sanitize equipment for, among other things, manufacturing pharmaceutical and biological products. Typical oxidizing substances used for such purposes are peroxides, chlorine or bromine containing compounds, such as sodium hypochlorite (e.g., bleach), calcium hypochlorite, sodium dichloroisocyanurate, 1-bromo-3-chloro-5,5-dimethylhydantoin (BCDMH), and hydrogen peroxide. Certain pharmaceutical and biological products are sensitive to residual or trace quantities of oxidizing substances and undergo degradation when exposed to these substances. Contamination of manufacturing equipment by oxidizing substances used for cleaning, decontamination, or sanitization may lead to degradation of pharmaceutical and biological products during manufacturing or storage. Degradation of pharmaceutical and biological products results in significant loss of product and unnecessary financial loss associated with successful manufacturing of otherwise viable product.

Known methods for detecting and measuring oxidizing substances include orthotolidine (OT), diethyl-p-phenylene diamine, or ferric ammonium sulfate/diethyl-p-phenylene diamine (DPD/FAS-DPD). These methods result in a colored yellow complex (OT) or pink complex (DPD) measured using a compactor tube or ultraviolet light. The limit of these methods is about 50 parts per billion or about 50 nanograms/mL free chlorine. Other methods include iodometric titration, which oxidizes iodide to iodine then measures iodine concentration by titration with thiosulfate and the end point detected visually using starch or potential differences. Another method is amperometric titration in which the sample is titrated with phenylarsine oxide and the end point detected by a decreased current. The quantitation limits of iodometric and amperometric titration are typically less than OT/DPD methods. Such methods are described in Standard Methods for the Examination of Water and Wastewater, 23^{rd} Edition, Published by American Water Works Association (AWWA, WEF, and APHA), 2017.

It was discovered that certain peptides are highly sensitive to oxidizing substances, including trace or residual amounts left behind after cleaning, decontaminating and sanitizing equipment. Manufacturing equipment having trace or residual levels of oxidizing substances leads to degradation of peptides during manufacture and loss of product. Therefore, a need exists for methods of detecting trace or residual levels of oxidizing substances with a quantitation limit lower than known methods.

### SUMMARY

In one aspect, an analytical method for detecting presence of oxidizing substances by peptide degradation may include mixing a peptide with a sample to create a test preparation, and detecting impurities of the peptide in the test preparation. The detected impurities indicate presence of oxidizing substances in the sample.

In another aspect, an analytical method for detecting presence of oxidizing substances by peptide degradation may include preparing a test preparation including a peptide and a sample in which the peptide degrades in the presence of oxidizing substances; detecting impurities of the peptide in the test preparation in which detected impurities indicate presence of oxidizing substances in the sample.

In one example, the method includes identifying the detected impurities of the peptide in the test preparation. In another example, the method includes measuring the amount of the detected impurities. In one example, detecting impurities of the peptide in the test preparation includes analyzing the test preparation using high performance liquid chromatography or ultrahigh performance liquid chromatography.

In any of the above aspects or examples, the peptide is vasopressin. In any of the above aspects or examples, the sample includes a rinse solution or a swab sample. In another example, the method includes collecting a sample from equipment previously exposed to a cleaning solution. Examples of cleaning solutions include chlorine-containing compounds, peroxide-containing compounds, or bromine-containing compounds. In any of the above aspects or examples, the oxidizing substances may include peroxide-containing compounds, chlorine-containing compounds, or bromine-containing compounds. Examples of chlorine or bromine-containing compounds include sodium hypochlorite, calcium hypochlorite, sodium dichloroisocyanurate, or 1-bromo-3-chloro-5,5-dimethylhydantoin (BCDMH). An example of a peroxide-containing compound is hydrogen peroxide.

In any of the above aspects or examples, the amount of oxidizing substances present in the sample is from about 0.05 ng/mL to about 100 ng/mL. In another example, the amount of oxidizing substances present in the sample is from about 0.125 ng/mL to about 50 ng/mL. In yet another example, the amount of oxidizing substances present in the sample is from about 0.5 ng/mL to about 25 ng/mL.

### BRIEF DESCRIPTION OF THE DRAWINGS

FIG. 1 shows a chromatogram of a control rinse sample solution containing 0 ng/mL available chlorine.

FIG. 2 shows an expanded view of a portion of FIG. 1.

FIG. 3 shows a chromatogram of a rinse sample solution containing 100 ng/mL available chlorine.

FIG. 4 shows an expanded view of a portion of FIG. 3.

FIG. 5 shows a chromatogram of a rinse sample solution containing 10 ng/mL available chlorine.

FIG. 6 shows an expanded view of a portion of FIG. 5.

FIG. 7 shows a chromatogram of a rinse sample solution containing 5 ng/mL available chlorine.

FIG. 8 shows an expanded view of a portion of FIG. 7.

FIG. 9 shows a chromatogram of a rinse sample solution containing 1 ng/mL available chlorine.

FIG. 10 shows an expanded view of a portion of FIG. 9.

FIG. 11 shows a chromatogram of a rinse sample solution containing 0.5 ng/mL available chlorine.

FIG. 12 shows an expanded view of a portion of FIG. 11.

FIG. 13 shows a chromatogram of a rinse sample solution containing 0.1 ng/mL available chlorine.

FIG. 14 shows an expanded view of a portion of FIG. 13.

FIG. 15 shows a chromatogram of a rinse sample solution containing 0.05 ng/mL available chlorine.

FIG. 16 shows an expanded view of a portion of FIG. 15.

FIG. 17 shows a chromatogram overlay of the rinse sample solutions illustrated in FIGS. 1, 3, 5, 7, 9, 11, 13, and 15.

FIG. 18 shows an expanded view of a portion of FIG. 17.

FIG. 19 shows a chromatogram of a swab sample control containing 0 ng/25 cm² available chlorine.

FIG. 20 shows an expanded view of a portion of FIG. 19.

FIG. 21 shows a chromatogram of a swab sample containing 1000 ng/25 cm² available chlorine.

FIG. 22 shows an expanded view of a portion of FIG. 21.

FIG. 23 shows a chromatogram of a swab sample containing 500 ng/25 cm² available chlorine.

FIG. 24 shows an expanded view of a portion of FIG. 23.

FIG. 25 shows a chromatogram of a swab sample containing 50 ng/25 cm² available chlorine.

FIG. 26 shows an expanded view of a portion of FIG. 25.

FIG. 27 shows a chromatogram of a swab sample containing 25 ng/25 cm² available chlorine.

FIG. 28 shows an expanded view of a portion of FIG. 27.

FIG. 29 shows a chromatogram of a swab sample containing 5 ng/25 cm² available chlorine.

FIG. 30 shows an expanded view of a portion of FIG. 29.

FIG. 31 shows a chromatogram of a swab sample containing 2.5 ng/25 cm² available chlorine.

FIG. 32 shows an expanded view of a portion of FIG. 31.

FIG. 33 shows a chromatogram of a swab sample containing 0.5 ng/25 cm² available chlorine.

FIG. 34 shows an expanded view of a portion of FIG. 33.

FIG. 35 shows a chromatogram of a swab sample containing 0.25 ng/25 cm² available chlorine.

FIG. 36 shows an expanded view of a portion of FIG. 35.

FIG. 37 shows a chromatogram overlay of the swab sample solutions illustrated in FIGS. 21, 23, 25, 27, 29, 31, 33 and 35.

FIG. 38 shows an expanded view of a portion of FIG. 37.

### DETAILED DESCRIPTION

Disclosed are systems and methods for detecting presence of oxidizing substances using peptide degradation. Certain peptides degrade and produce impurities when exposed to oxidizing substances. The systems and methods include preparing a test preparation. The test preparation may be prepared by mixing a peptide with sample. The systems and methods may be utilized to detect presence of peptide impurities in the test preparation to advantageously indicate presence of oxidizing substances in samples. The systems and methods allow for detection of trace or residual amounts of oxidizing substances. Thus, the systems and methods according to the present disclosure improve upon prior methodologies that provide detection of oxidizing substances only at higher amounts. For example, the systems of methods according to the present disclosure may detect oxidizing substances from about 0.05-100 ng/mL.

In one example, a sample is collected from equipment having been previously exposed to cleaning solution. The sample may be in the form of a rinse solution collected from the equipment. In another example, the sample may be a swab sample taken from the surfaces of the equipment. It will be understood that other methods of collecting a sample may be used.

The equipment may be any manufacturing equipment used for biological or pharmaceutical manufacturing. The examples are also not limited to manufacturing equipment and may be any device or surface that comes into contact with a biological or pharmaceutical product that could be a source of contamination. Further, the methods of the present disclosure have applicability in other fields outside of biological or pharmaceutical products in which presence of oxidizing substances may be of interest. For example, the methods of the present disclosure may be used in fields in which oxidizing substances are measured or controlled.

In one example, the oxidizing substances include chlorine or bromine-containing compounds. In other examples, the oxidizing substances form part of a cleaning solution used to clean, disinfect, or sanitize equipment or surfaces coming into contact with products, such as biological or pharmaceutical products. Examples of oxidizing substances include hydrogen peroxide, sodium hypochlorite (e.g., bleach), calcium hypochlorite, sodium dichloroisocyanurate (e.g., Dichlor) and 1-bromo-3-chloro-5,5-dimethylhydantoin (BCDMH).

Peptides suitable for use in the present disclosure are those which degrade in the presence of oxidizing substances and the impurities produced from degradation of the peptide may be detected. In another example, the peptide is selected such that it degrades in the presence of trace or residual amounts of oxidizing substances. In yet another example, the peptide is selected such that degradation of the peptide creates impurities capable of being detected. Examples of peptides include vasopressin and lypressin. Other peptides that degrade in the presence of oxidizing substances to impurities capable of detection are also contemplated by the present disclosure.

In one example, a test preparation is prepared by combining the peptide with the sample. Techniques for combining include mixing, sonication, or other suitable methods to expose the peptide to any potential oxidizing substances contained in the sample. Diluents and other ingredients, such as water, may be used in addition to the peptide and sample to prepare the test preparation for analysis and detection. In one example, the test preparation is allowed to react for a period of time, e.g., four hours, at ambient temperature. Other conditions may be used to provide sufficient time and conditions allowing for the peptide to be exposed with the sample in the test preparation.

In one embodiment, peptide, sample, test preparation, or combination thereof may be suitably diluted if necessary, e.g., to account for limitations of analytical equipment or to achieve a desired concentration. The diluent may be any suitable diluent, such as those described herein.

In one example, the test preparation is inputted or loaded into suitable instrumentation for detection. Examples of detection devices include high performance liquid chromatography (HPLC) or ultra-high performance liquid chromatography (UHPLC). Additional solutions may be prepared as needed to conduct analysis of the test preparation. For example, diluents may be prepared, such as acetic acid diluents, buffers for mobile phase, such as ammonium phosphate, acetonitrile, or other suitable buffers, stock matrix, such as sodium lactate with sodium chloride, to matrix match sample and standards for accurate detection may be used for the analysis using HPLC or UHLPC.

In various embodiments, working solutions of peptide may be used, such as specific concentrations for combining with sample, and resolution solutions for calibrating the HPLC or UHPLC instrumentation. Examples include 0.4 U/mL vasopressin for the working solution and 336 ng/mL vasopressin and 400 ng/mL lypressin for the resolution solution to assess suitability of the instrumentation. Blank diluent may also be used, such as water or other suitable diluents. Other suitable peptides and amounts and concentrations may be used for working solutions and resolutions solutions as determined by the peptide chosen for use in the systems and methods of the present disclosure.

The systems and methods described herein may include generation and/or comparison of detected peptide impurities with standards for quantitative determination. Those having skill in the art will appreciate that analysis may utilize more than one standard solution for concentration. For example, if a concentration range is wide, multiple standard solutions may be used to generate appropriate calibration data. For example, standard spike solutions of chlorine and bromine-containing compounds may be prepared. In one example, chlorine or bromine solutions having from about 0.05 to about 100 nanograms/mL available chlorine or bromine may be used in the present disclosure. In another embodiment, chlorine or bromine solutions having from about 0.125 to about 50 nanograms/mL available chlorine or bromine are used in the present disclosure. In yet another example, chlorine or bromine solutions having from about 0.5 to about 25 nanograms/mL available chlorine or bromine are used in the present disclosure. The spike solutions may be prepared in multiples at various increments over suitable ranges. For example, spike solution may be prepared at intervals of factors of 2 through 10, inclusive of decimal increments, or other suitable factors.

Operation of HPLC or UHPLC columns is known in the art. Analysis may occur at a suitable parameters and operating conditions based on the peptide. Suitable conditions of mobile phases, flow rates, detector wavelength, scan range, column temperature and injection volume may be determined as known in the art. Examples include ammonium phosphate as one mobile phase and ammonium phosphate and acetonitrile as a second mobile phase. Exemplary flow rate includes 0.6 mL/min, and other flow rates may be used in the present disclosure. Exemplary detector wavelengths include 220 nm and a scan range of 214-350, while other suitable wavelengths and scan ranges may be used in the present disclosure. Examples of column temperature include 35° C. ± 2° C., while other temperatures and temperature ranges may be used in the present disclosure. Examples of injection volumes include 500 microliters, and other suitable injections volumes may be used in the present disclosure.

In various embodiments, the order of injection into the HPLC or UHPLC instrumentation includes a blank, such as diluent, followed by a resolution solution, standards and the test preparation. In one example, additional standards and replicates of the same standard or sample may be used. The instrumentation is capable of detecting impurities of the peptide resulting from exposure and reaction with oxidizing substances found in the sample. In one example, the instrumentation may detect impurities of peptide resulting from concentrations of oxidizing substances from about 0.05 ng/mL. Thus, the present disclosure allows for detection of trace or residual amounts of oxidizing substances present in the sample through use of degradation of peptides and detection of impurities created from peptide degradation.

In one embodiment, a system of detecting presence of oxidizing substances comprises a kit including one or more peptides and other standard spike solutions described herein with respect to detecting, identifying, and quantifying peptide impurities. For example, the kit may include peptide solutions, diluents, spike solutions of varying concentration, mobile phase buffers, or combinations thereof.

Detection of peptide impurities may be determined by comparison to peptide solutions, such as working solution standards. Presence of impurity peaks related to oxidized degradants of the peptide from the HPLC or UHPLC results indicate presence of oxidizing substances in the test preparation and sample. In addition, the present disclosure contemplates identifying the impurities detected by HPLC or UHPLC and quantifying the impurities. Impurity peak locations and identity may vary depending on the peptide used. In addition, the quantity of peptide impurities identified may vary depending on the peptide, the amount of oxidizing substances in the test preparation, or other operating parameters and conditions.

### Example - Determination of Oxidative Compounds by Peptide Degradation Using Ultra High Performance Liquid Chromatography

Oxidative compounds react with peptides to create oxidative impurities, which can be detected using UHPLC. Presence of oxidative impurities of vasopressin was determined from samples of rinse water or swabs as described below.

The following solutions were prepared. Multiples of all solutions (e.g., reagents, test solutions, standards, and samples) may be prepared. Fractions of solutions may also be prepared except for sample preparations and quantitative standards.

Preparation of Solutions. 0.25% Acetic Acid Diluent was prepared by diluting 5 ml glacial acetic acid to 2000 ml with water. The solution was filtered using 0.2 micrometer filter (preferably polyvinylidene fluoride (PVDF)). Mobile Phase Buffer (6.0 g/L ammonium phosphate, pH 3.0) was prepared by dissolving 10.0 g ammonium phosphate in 950 ml water. The pH was adjusted to 3.0 ± 0.1 using phosphoric acid (e.g., about 36 ml was needed). Solution was diluted to 2000 ml with water and pH was measured. Buffer was filtered using 0.2 micrometer filter (preferably Nylon or PVDF). Mobile Phase A (3.0 g/L Ammonium phosphate) was prepared by diluting 500 ml of Mobile Phase Buffer with 500 ml water and filtering using a 0.2 micrometer filter (preferably Nylon of PVDF). Mobile Phase B was prepared by mixing 500 ml Mobile Phase Buffer with 500 ml acetonitrile and filtering using a 0.2 micrometer filter (preferably Nylon of PVDF). Stock Matrix (30 mM sodium lactate, 9% sodium chloride, pH 3.6) was prepared by dissolving 3.36 g sodium lactate and 90 g sodium chloride in 800 ml water. The pH can be adjusted to pH of 3.6 ± 0.1 as needed using sodium hydroxide or hydrochloric acid. Solution was diluted to 1000 ml with water.

Preparation of Suitability Solutions. Stock Vasopressin (0.0084 mg/ml vasopressin) was prepared by dissolving and diluting one vial of vasopressin USP reference standard to 200 ml with 0.25% Acetic Acid Diluent. Resulting solution contains approximately 4.5 to 4.7 units/mL. Resolution Stock (0.01 mg/ml lypressin) was prepared by dissolving and diluting 1 vial of lypressin USP reference standard to 200 ml with 0.25% Acetic Acid Diluent. The following Working Solutions were prepared by diluting with water as shown in Table 1:

TABLE 1

Working Solutions

| Solution | Stock Standard Vasopressin | Resolution Stock | Stock Matrix | Final volume | Approximate Concentration (Based on a vial of vasopressin USP reference standard containing 926 Units (1.68 mg)/vial |
|---|---|---|---|---|---|
| | mL | mL | mL | mL | |
| Vasopressin Solution | 44.0 | 0.0 | 50.0 | 500.0 | 0.4 U/mL Vasopressin |
| Resolution Solution | 2.0 | 2.0 | 5.0 | 50.0 | 336 ng/mL Vasopressin 400 ng/mL Lypressin |
| Blank/Sample Diluent | N/A | N/A | 100.0 | 1000.0 | N/A |

Vasopressin solution was prepared by mixing 44.0 ml stock vasopressin with 50.0 ml Stock Matrix, and the balance water to achieve a final volume of 500.0 ml. The vasopressin solution contained approximately 0.4 U/mL. Resolution Solution was prepared by mixing 2.0 ml stock vasopressin, 2.0 ml Resolution Stock, 5.0 ml Stock Matrix and the balance water to achieve a final volume of 50.0 ml. Blank/Sample Diluent was prepared by diluting 100.0 ml Stock Matrix to 1000 ml with water and filtering using a 0.2 micrometer filter.

Preparation of Standards. 1% Sodium Hypochlorite Stock Solution was prepared from a stock calcium hypochlorite, sodium hypochlorite, and sodium dichloroisocyanurate (Dichlor) solutions based on the certificate of analysis or titration assay value. For example, if the certificate of analysis value is 6% (w/w), then 16.67 g of a 6% sodium hypochlorite solution is needed per 100 ml (100 ml x (1%/6%) = 16.67).

From the stock solution, 1000 µg/mL solutions were prepared as described above. Preparations may be modified based on the available chlorine determined per titration as in the following Equation 1: 1000 µg/mL Stock Solution Volume Needed, mL

$$\frac{1000 \text{μ} \text{} \text{g} / \text{mL}}{\% \text{w} / \text{V}} \times \frac{100.0}{1000000 \text{μ} \text{} \text{g}} \times \text{Final} \text{volume} \left(\text{mL}\right)$$

From the 1000 µg/mL solutions, spike solutions having the concentrations provided in Table 2 were prepared by dilution as described above. Due to the relative instability of available chlorine in water, these solutions should be prepared the same day of the test solution preparations.

TABLE 2

Chlorine Spike Solutions

| Spike Solution | Sodium Hypochlorite Stock Solution | | Final Volume w/ water | Concentration |
|---|---|---|---|---|
| | µg/mL | mL Stock Solution | mL | µg/mL Available Chlorine |
| 50 | 1000 | 5.0 | 100 | 50 |
| 25 | 1000 | 5.0 | 200 | 25 |
| 5 | 1000 | 0.5 | 100 | 5 |
| 25 | 1000 | 0.5 | 200 | 2.5 |
| 1.0 | 50 | 2.0 | 100 | 1.0 |
| 0.5 | 50 | 2.0 | 200 | 0.5 |
| 0.25 | 50 | 1.0 | 200 | 0.25 |
| 0.125 | 50 | 0.5 | 200 | 0.125 |
| 0.05 | 10 | 0.5 | 100 | 0.05 |
| 0.025 | 10 | 0.5 | 200 | 0.025 |

The following Standard Solutions were prepared as shown in Table 3

TABLE 3

Standard Solutions

| Standard Solution | Spike | | Water | Vasopressin Solution | Available Chlorine concentration |
|---|---|---|---|---|---|
| | Spike Solution | µL | mL | mL | ng/mL sample |
| Blank | Water | 10.0 | 5.0 | 5.0 | 0 |
| 5 | 2.5 | 10.0 | 5.0 | 5.0 | 5 |
| 2 | 1.0 | 10.0 | 5.0 | 5.0 | 2 |
| 1 | 0.5 | 10.0 | 5.0 | 5.0 | 1 |
| 0.5 | 0.25 | 10.0 | 5.0 | 5.0 | 0.5 |
| 0.25 | 0.125 | 10.0 | 5.0 | 5.0 | 0.25 |

Test Solution Preparation. Test solutions were prepared by spiking chlorine spike solutions into a vasopressin solution. The solutions were then allowed to react for four (4) hours before diluting to final concentration (approximately 0.2 U/mL vasopressin) with sample diluent. Rinse sample test solutions were prepared as follows: (1) pipette water; (2) pipette chlorine spike; (3) allow to mix; (4) add vasopressin spike; (5) allow to mix and react for four (4) hours at ambient temperature. Table 4 shows the various rinse sample test solutions.

TABLE 4

Rinse Sample Test Solutions

| Sample | Spike | | Water | 0.4 u/mL Vasopressin | Available Chlorine concentration |
|---|---|---|---|---|---|
| | Solution from Table 2 | µL | mL | mL | ng per mL sample |
| TS-Blank | 100 | 5 | 5.0 | 5.0 (Sample Diluent) | --- |
| TS-Control | Water | 5.0 | 5.0 | 5.0 | 0 |
| TS-100 | 100 | 5.0 | 5.0 | 5.0 | 100 |
| TS-10 | 10 | 5.0 | 5.0 | 5.0 | 10 |
| TS-5 | 5 | 5.0 | 5.0 | 5.0 | 5 |
| TS-1 | 1 | 5.0 | 5.0 | 5.0 | 1 |
| TS-0.5 | 0.5 | 5.0 | 5.0 | 5.0 | 0.5 |
| TS-0.1 | 0.1 | 5.0 | 5.0 | 5.0 | 0.1 |
| TS-0.05 | 0.05 | 5.0 | 5.0 | 5.0 | 0.05 |

The concentrations are expressed as ng of available chlorine per mL rinse sample per Equation 2: Rinse Sample Concentration as Available Chlorine per mL Rinse Sample

$$\frac{\frac{\text{μ} \text{g}}{\text{mL}} \text{spike} \text{solution} \times μ \text{L} \text{spike} \times \frac{1 \text{mL}}{1000 \text{μ} \text{L}} \times \frac{1000 \text{ng}}{1 \text{μ} \text{g}}}{5 \text{mL} \text{sample}}$$

Swab sample test solutions were prepared in a TOC vial as follows: (1) add specified volume of vasopressin 0.4 U/mL solution; (2) to a single swab, spike the swab with the volume and solution specified; (3) immediately place swab into TOC vial containing 0.4 U/mL vasopressin solution; (4) sonicate vial for two (2) mins, followed by mixing using a vortex mixer; (5) allow to sit at room temperature for four hours; (5) after four hours, add 5.0 mL of sample diluent. Table 5 shows the swab sample test solutions prepared.

TABLE 5

Swab Sample Test Solutions

| Sample | TOC Vial Solution | | Spike to swab | | Available Chlorine |
|---|---|---|---|---|---|
| | Solution | Volume | Spike Solution (Table 2) | µL | ng/25 cm² |
| TS-Blank swab | Sample Diluent | 5.0 | 100 | 5 | --- |
| TS-Control swab | 0.4 U/mL | 5.0 | Water | 5 | N/A |
| TS-1000 swab | 0.4 U/mL | 5.0 | 1000 µg/mL solution (from above) | 5 | 1000 |
| TS-100 swab | 0.4 U/mL | 5.0 | 100 | 5 | 500 |
| TS-10 swab | 0.4 U/mL | 5.0 | 10 | 5 | 50 |
| TS-5 swab | 0.4 U/mL | 5.0 | 5 | 5 | 25 |
| TS-1 swab | 0.4 U/mL | 5.0 | 1 | 5 | 5.0 |
| TS-0.5 swab | 0.4 U/mL | 5.0 | 0.5 | 5 | 2.5 |
| TS-0.1 swab | 0.4 U/mL | 5.0 | 0.1 | 5 | 0.50 |
| TS-0.05 swab | 0.4 U/mL | 5.0 | 0.05 | 5 | 0.25 |

The concentrations are expressed as available chlorine per 25 cm² using the formula presented in Equation 3: Swab Sample Concentration as Available Chlorine per 25 cm²

$$\frac{\frac{\text{μ} \text{g}}{\text{mL}} \text{spike} \text{solution} \times μ \text{L} \text{spike} \times \frac{1000 \text{ng}}{\text{μ} \text{g}} \times \frac{1 \text{mL}}{1000 \text{μ} \text{L}}}{25 {\text{cm}}^{2}}$$

Test solution replicates were prepared as shown in Table 6. For purposes of this Example, a replicate was considered an individual preparation of test solution.

TABLE 6

Test Solution Replicates

| Table 6 Solutions | Replicates | Table 7 Solutions | Replicates |
|---|---|---|---|
| TS-Blank | 1 | TS-Blank swab | 1 |
| TS-Control | 1 | TS-Control swab | 3 |
| TS-100 | 1 | TS-1000 swab | 3 |
| TS-10 | 1 | TS-100 swab | 3 |
| TS-5 | 1 | TS-10 swab | 3 |
| TS-1 | 1 | TS-5 swab | 3 |
| TS-0.5 | 1 | TS-1 swab | 3 |
| TS-0.1 | 1 | TS-0.5 swab | 3 |
| TS-0.05 | 1 | TS-0.1 swab | 3 |
| | | TS-0.05 swab | 3 |

Rinse and swab samples may also be prepared using the following procedures. Rinse Sample Preparation was prepared by mixing 4.0 ml of rinse sample with 4.0 ml Vasopressin Solution in a suitable glass container. Solution then sat at room temperature for not less than four (4) hours. Swab Sample was prepared by adding swabs to a TOC vial containing 4.0 ml Vasopressin Solution. The swabs and Vasopressin Solution were sonicated using an ultrasonic booth for five (5) minutes, then mixed using a vortex mixer. Solution was allowed to sit at room temperature for not less than four (4) hours. After four hours, 4.0 ml of Sample Diluent was added to the vial.

Assay Procedure. Table 7 illustrates the approximate conditions for the HPLC column.

TABLE 7

HPLC Column Conditions

| Analytical Column | YMC Triart C18 ExRS 100 × 2.0 mm TAR08SP9-1002PT; 1.9 um P/N | | |
|---|---|---|---|
| Mobile Phase A | Ammonium Phosphate 3.0 g/L | | |
| Mobile Phase B | 50% 6.0 g/L ammonium phosphate 50% Acetonitrile | | |
| Gradient Profile | Time | % A | % B |
| | 0.00 | 85.0 | 15.0 |
| | 10.00 | 85.0 | 15.0 |
| | 20.00 | 15.0 | 85.00 |
| | 28.00 | 15.0 | 85.00 |
| | 28.01 | 85.0 | 15.0 |
| | 33.00 | 85.0 | 15.0 |
| Flow Rate | 0.6 mL/min. | | |
| Detector Wavelength 60 mm (or larger) flow cell required | 220 nm | | |
| PDA Scan range, nm | 214-350 | | |
| Column Temperature | 35° C. ± 2° C. | | |
| Injection Volume | 500 µL | | |
| Auto sampler Temperature | Set to 5° C. | | |
| Needle Wash (Recommended) | 80/20 Water/Acetonitrile | | |
| Seal Wash | 80/20 Water/Acetonitrile | | |
| Run time | 33 minutes | | |

Once the HPLC system reaches equilibrium at initial conditions and a stable baseline is achieved, solutions were injected in the following order as shown in Table 8.

TABLE 8

Order of Solution Injection
Solution Replicates Chromatogram

| Conditioning (Resolution solution) | 2 or more, as necessary | N/A |
|---|---|---|
| Blank | 1 | |
| Resolution solution | 1 | |
| Standard-5 | 1 | |
| Standard -2 | 1 | |
| Standard-1 | 1 | |
| Standard-0.5 | 1 | |
| Standard-0.25 | 1 | |
| Standard-1 | 6 | |
| Sample | 1 each | |
| Standard 1 | 1 | N/A |

Rinse sample test solutions were prepared per Table 4 as single replicates as shown in Table 8. Each rinse sample test solution replicate was injected in triplicate. Each replicate injection was from a separate vial.

Swab sample test solutions were prepared per Table 5 as triple replicates as shown in Table 8. Each replicate was injected s a single injection.

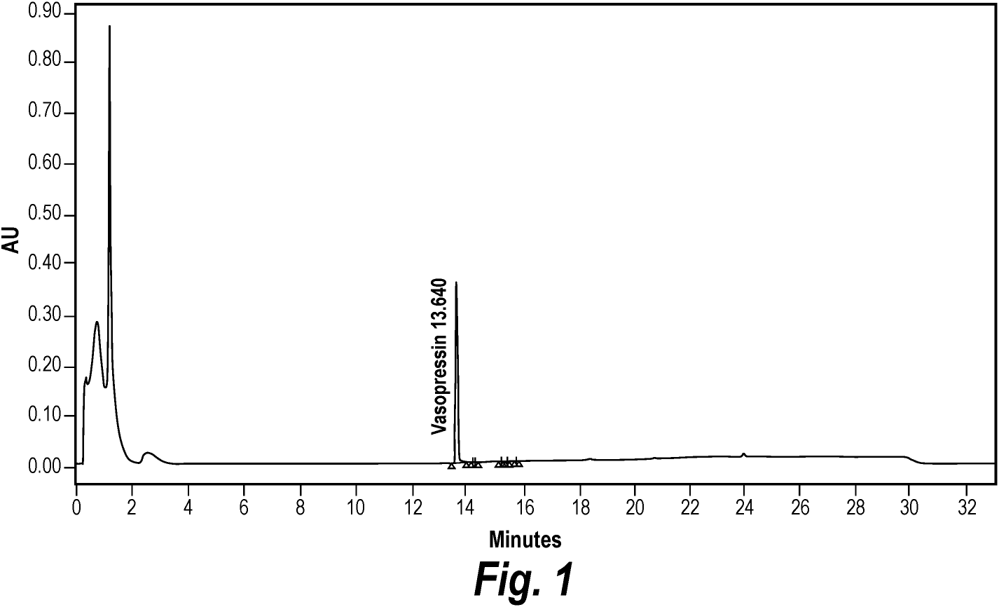

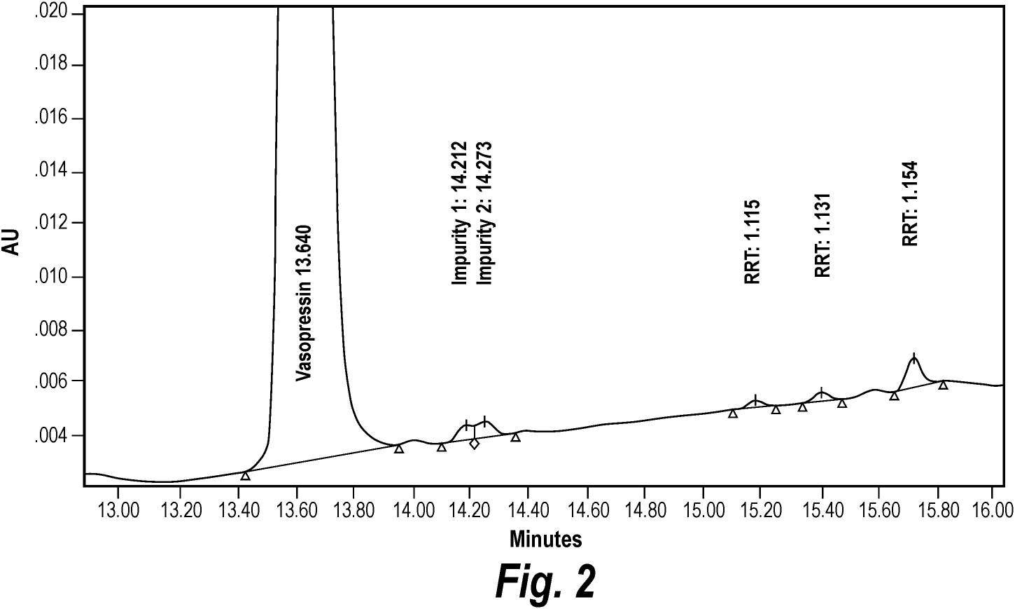

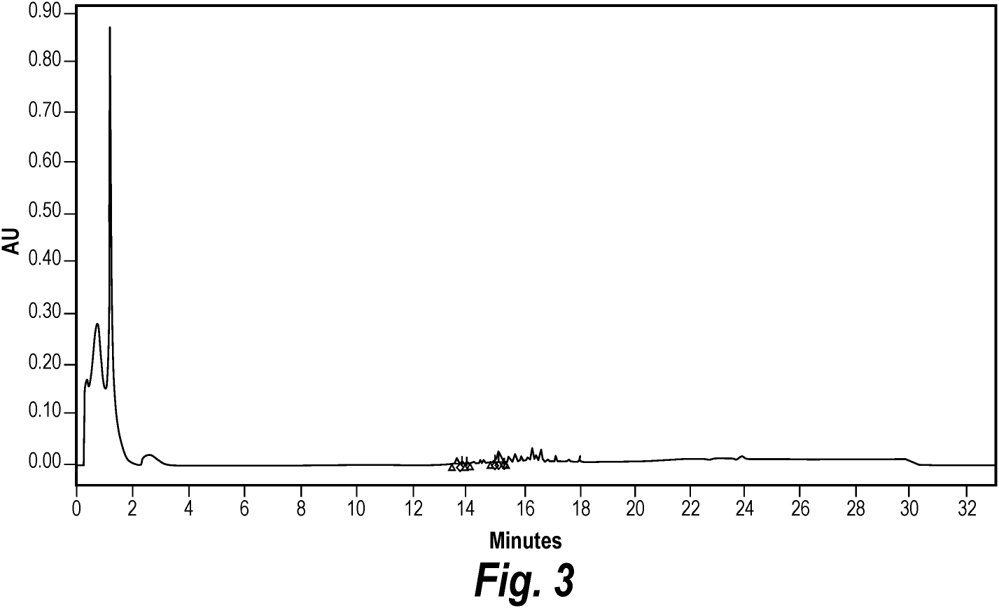

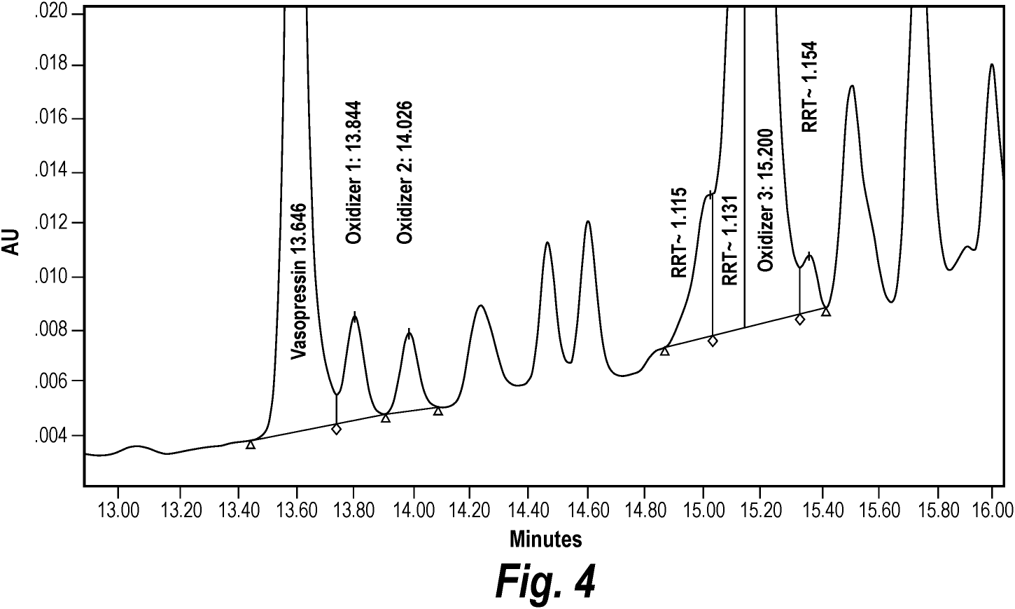

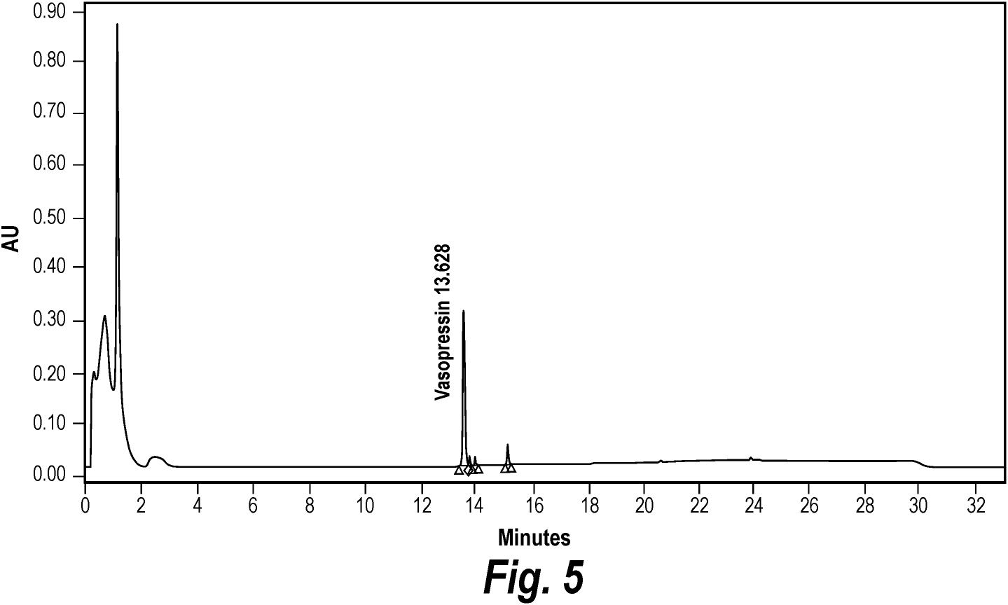

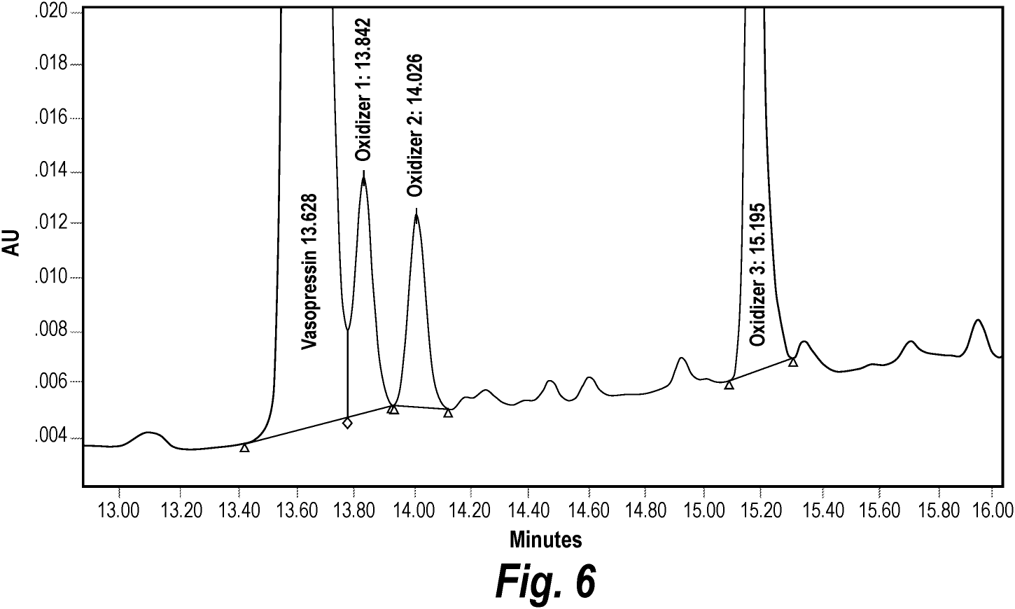

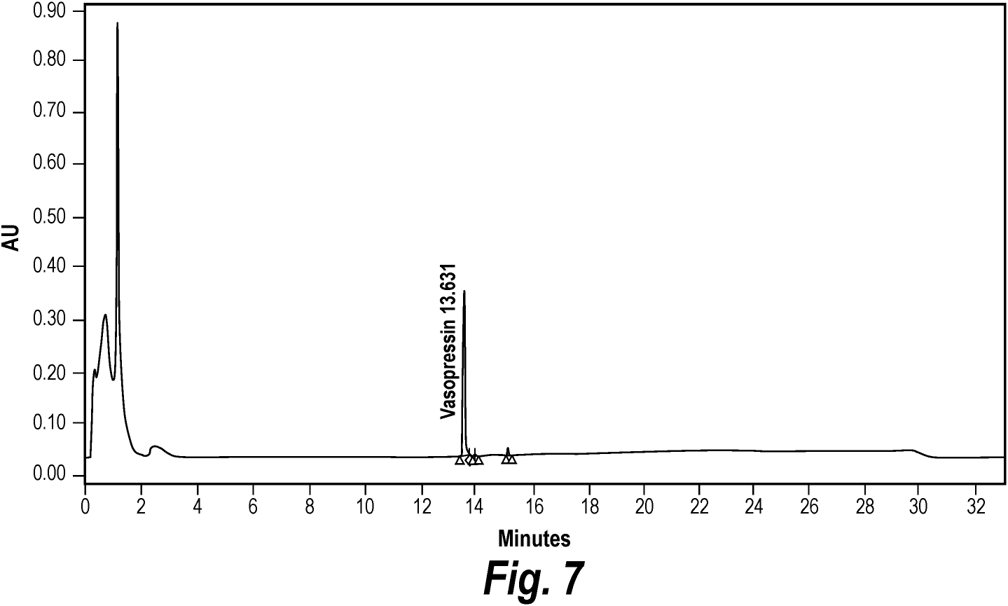

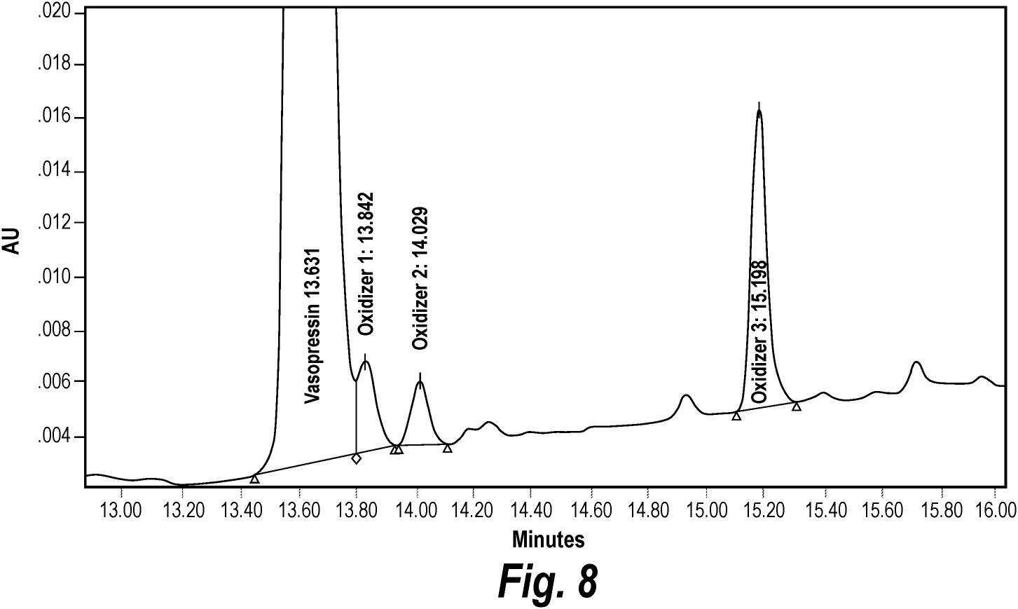

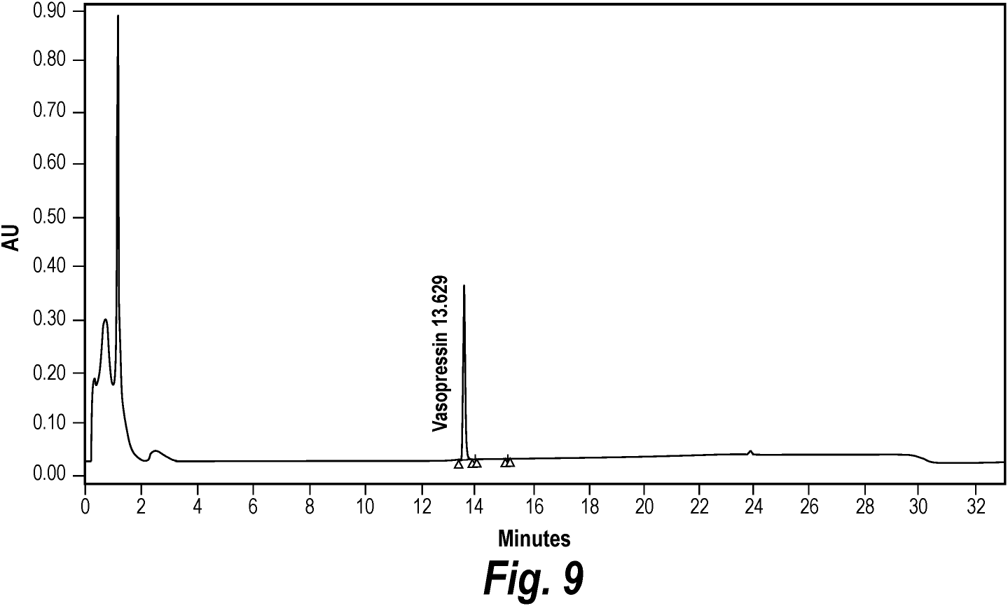

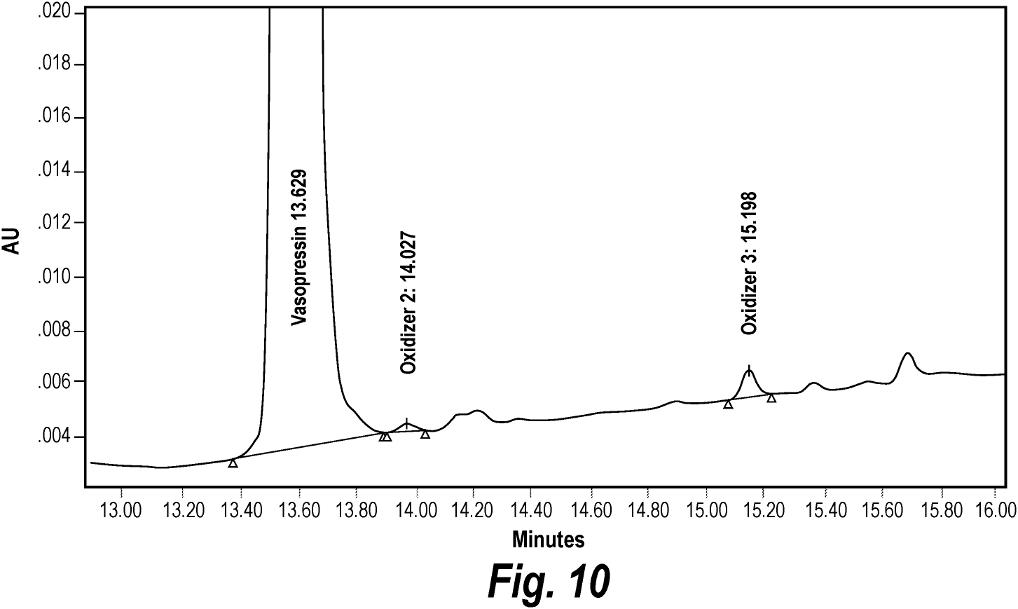

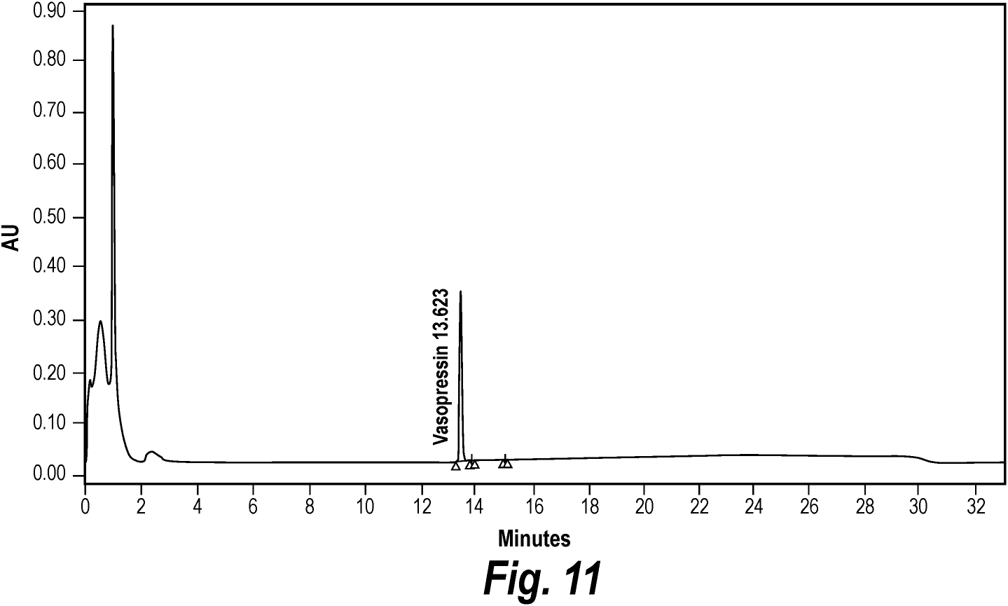

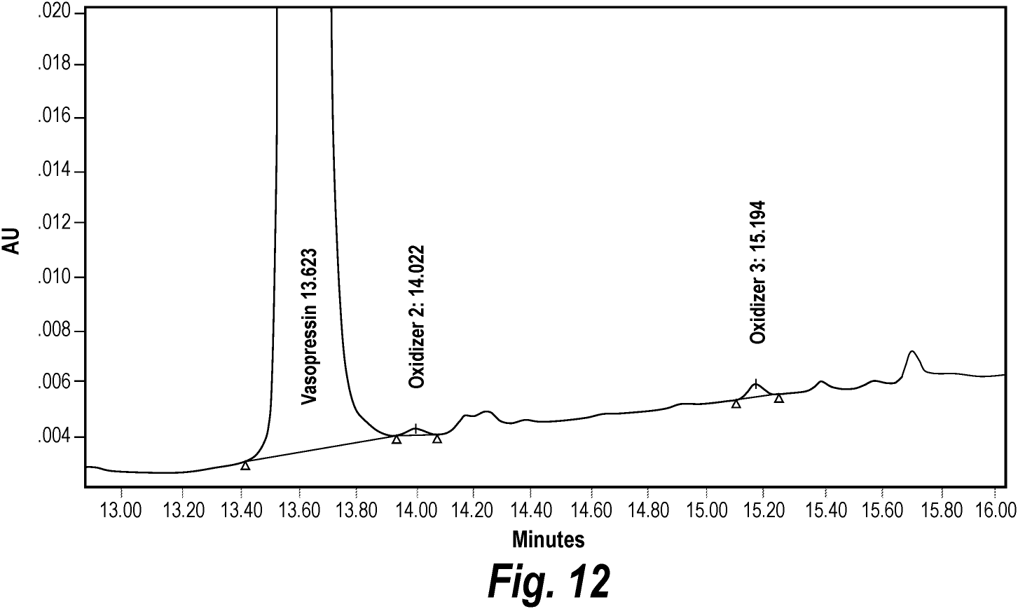

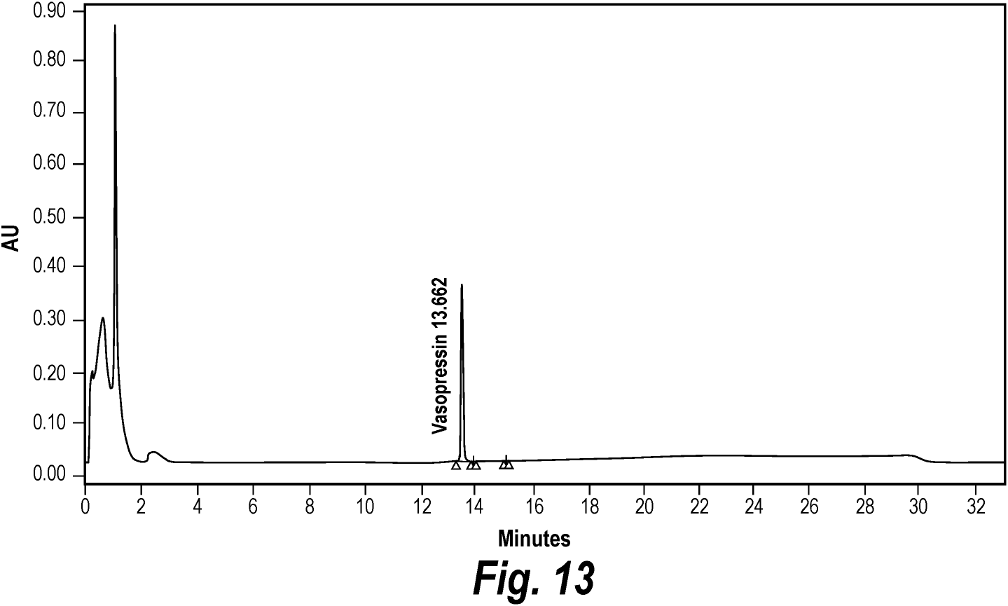

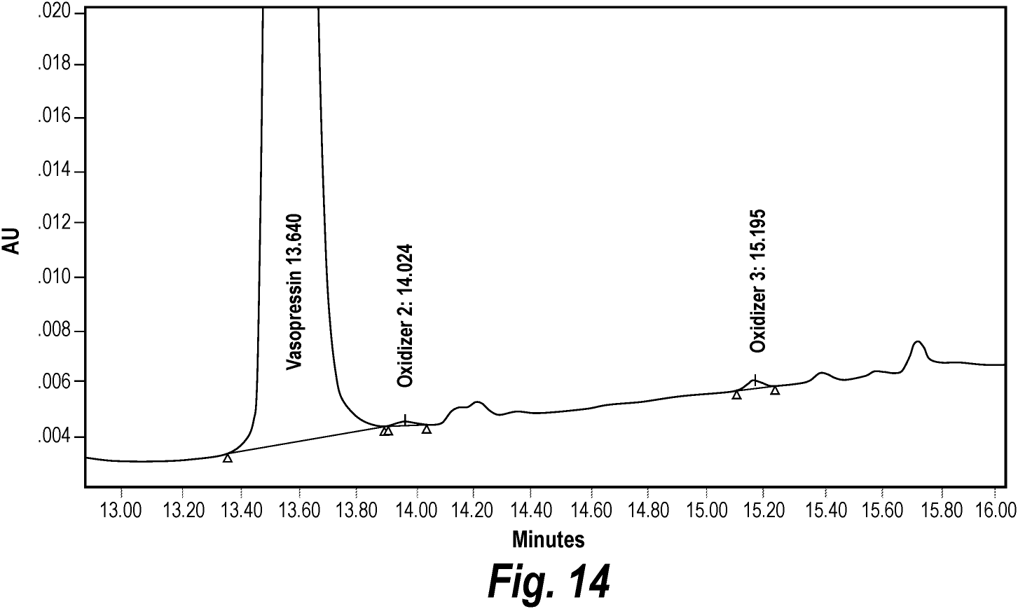

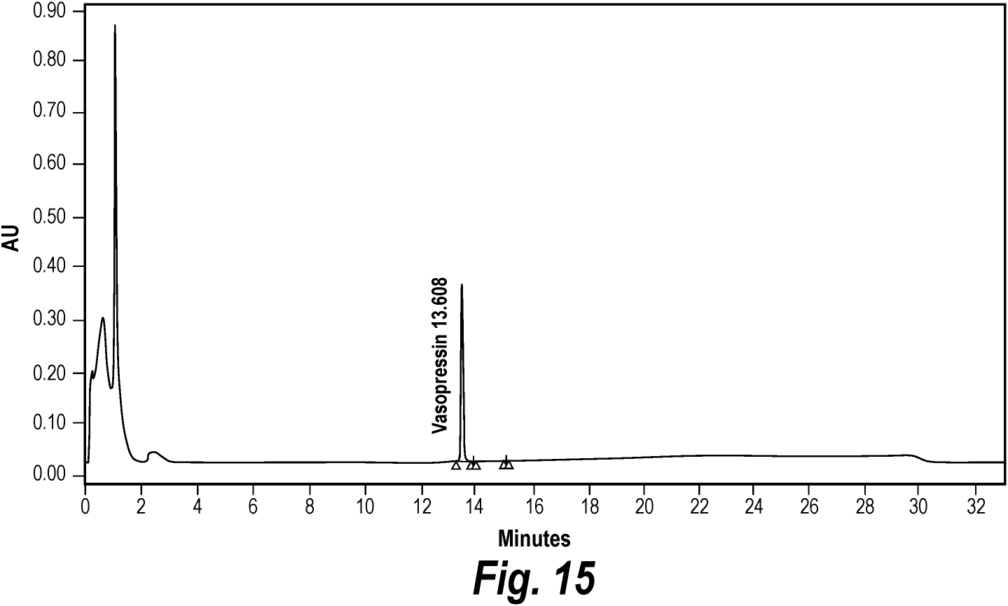

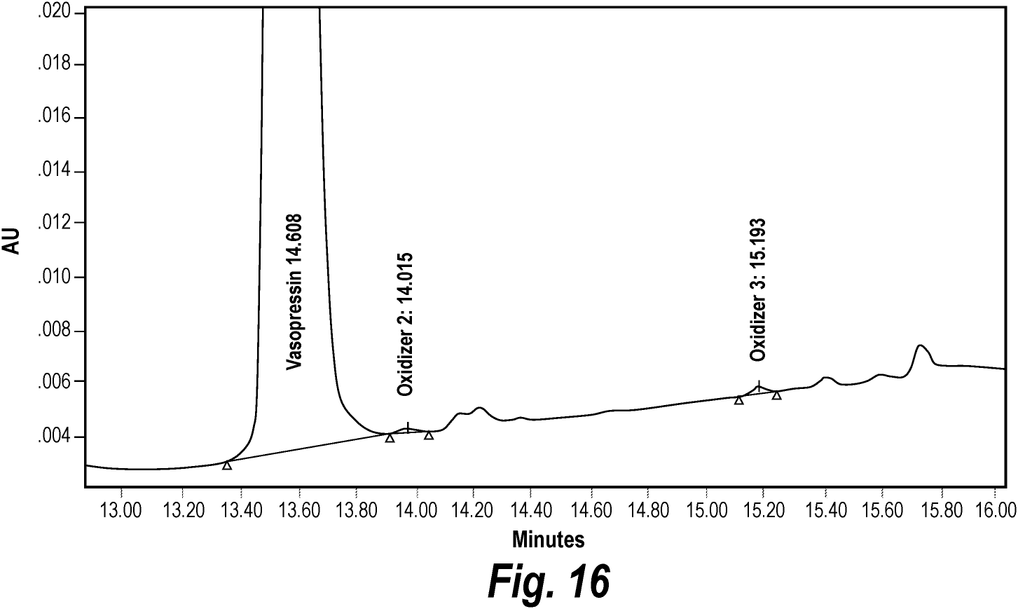

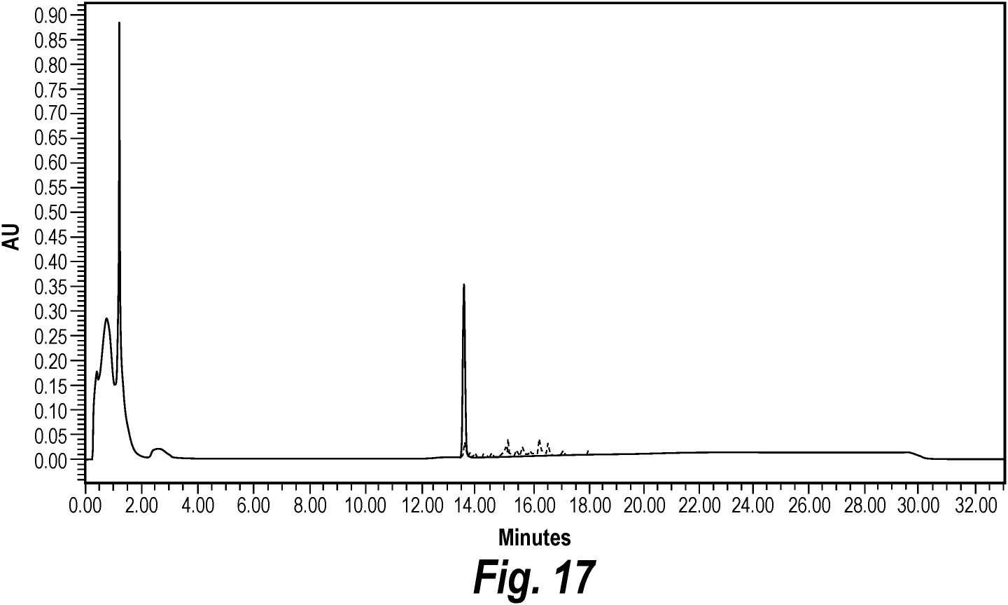

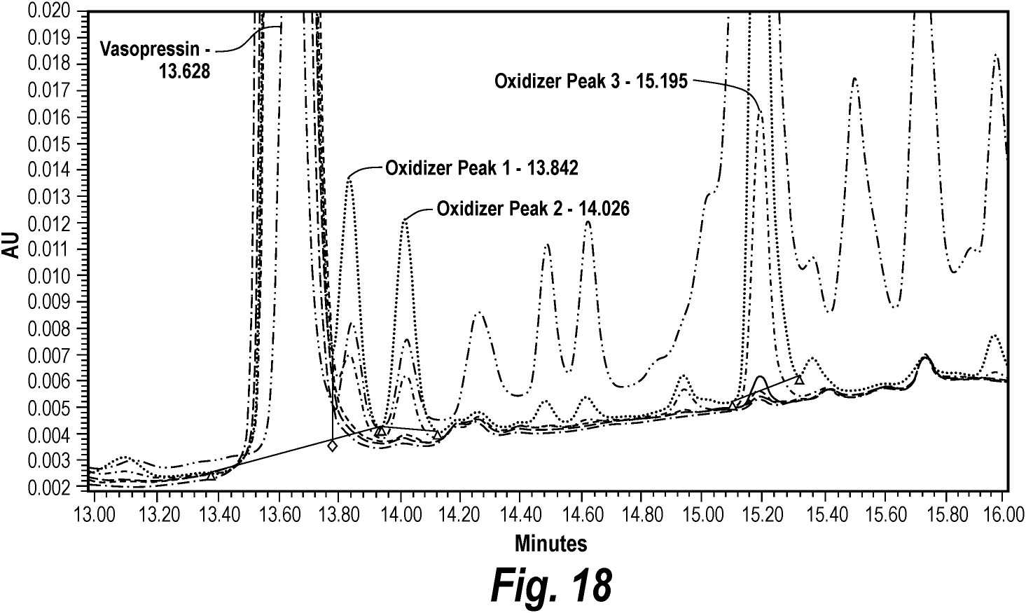

FIGS. 1-18 show the results of conducting HPLC analysis on control, and rinse sample solutions containing 100 ng/mL, 10 ng/mL, 5 ng/mL, 1 ng/mL, 0.5 ng/mL, 0.1 ng/mL, and 0.05 ng/mL available chlorine. Each chromatogram illustrates impurity peaks of vasopressin at about 13.7, about 13.95, and about 15.2 minutes. These figures illustrate the method of the present disclosure may detect trace or residual amounts of oxidizing substances as low as 0.05 ng/mL.

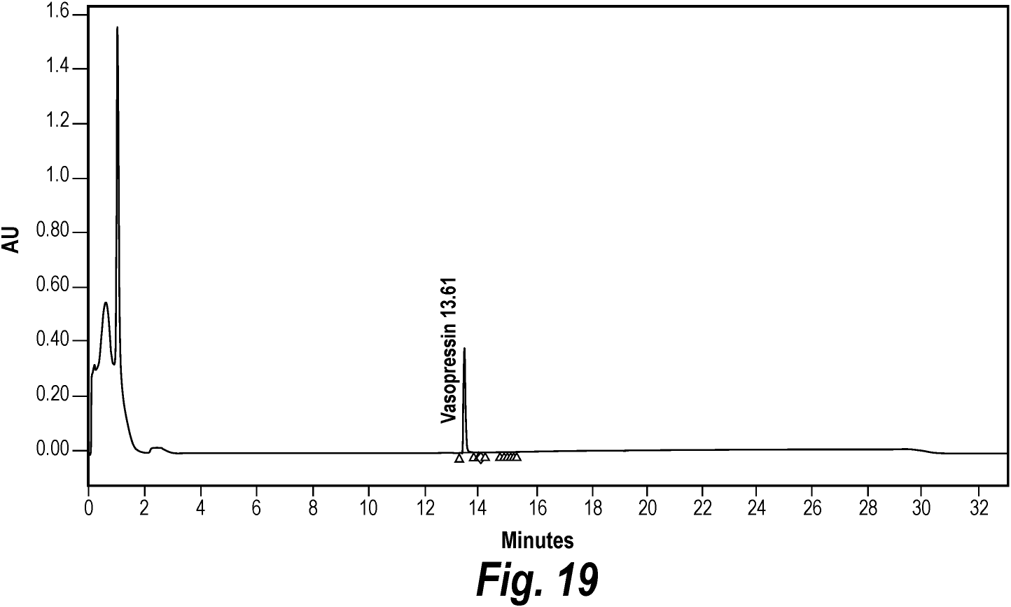

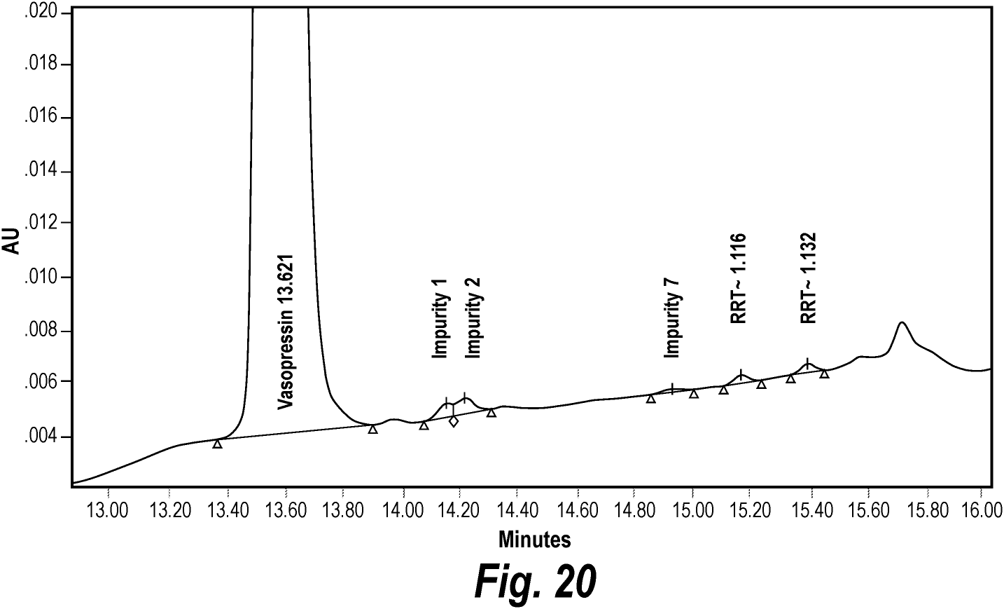

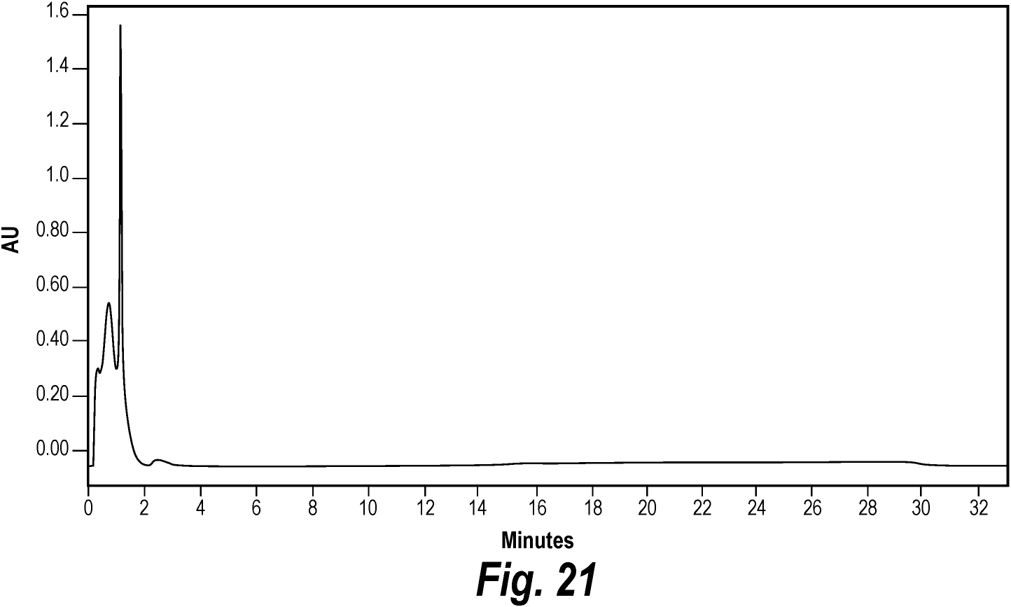

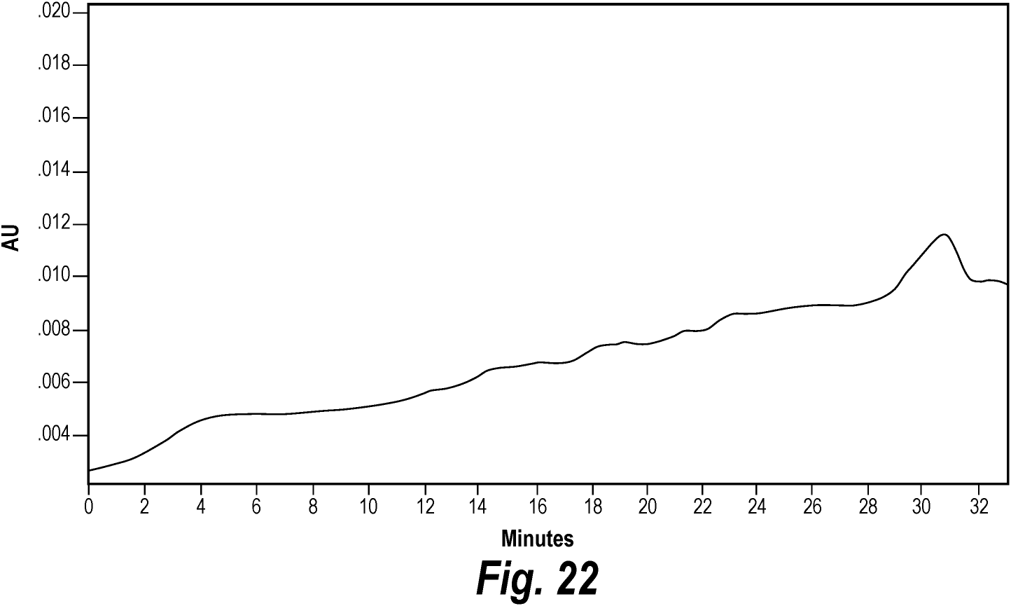

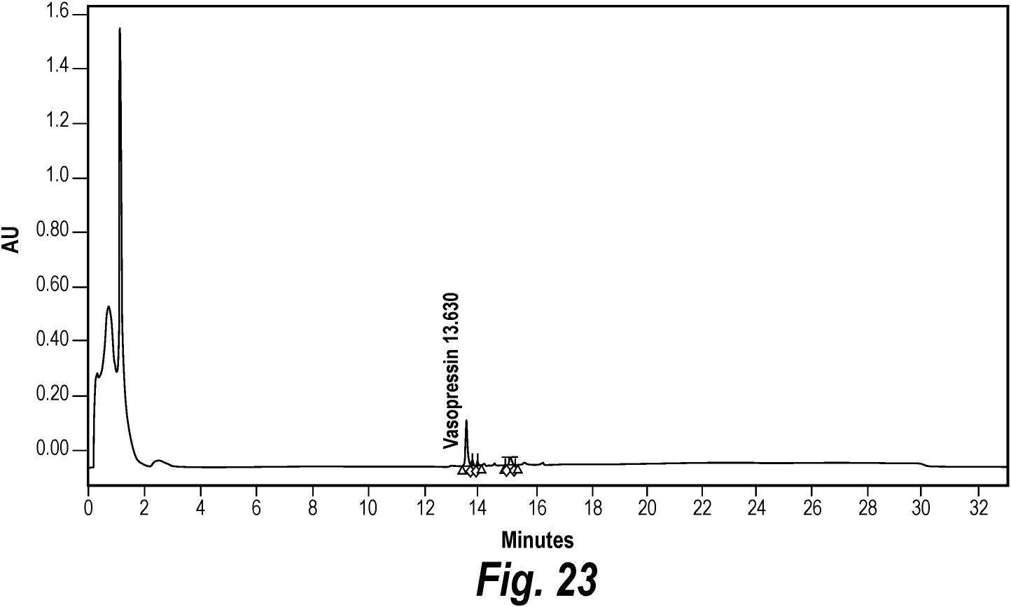

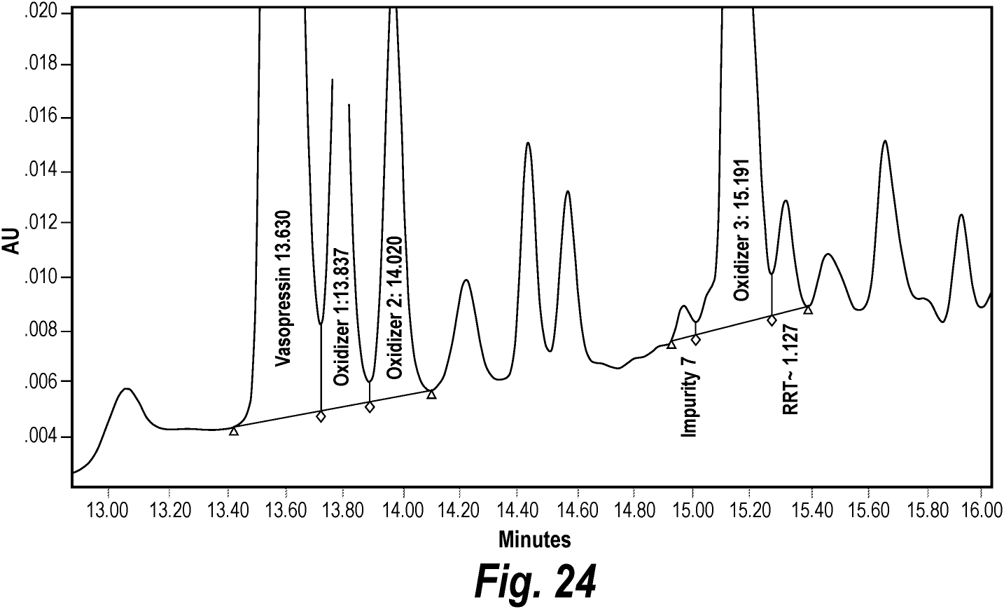

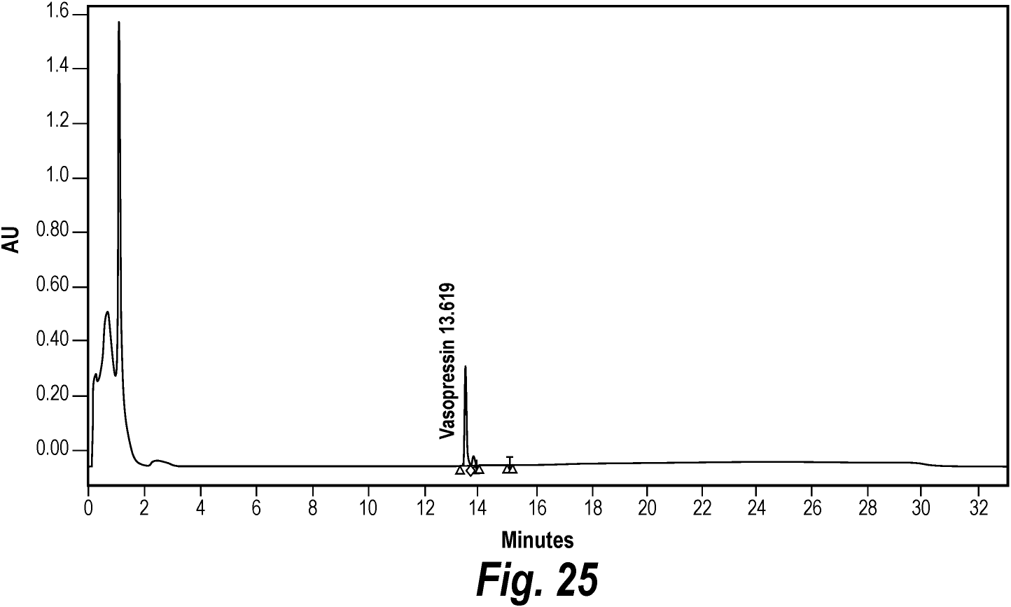

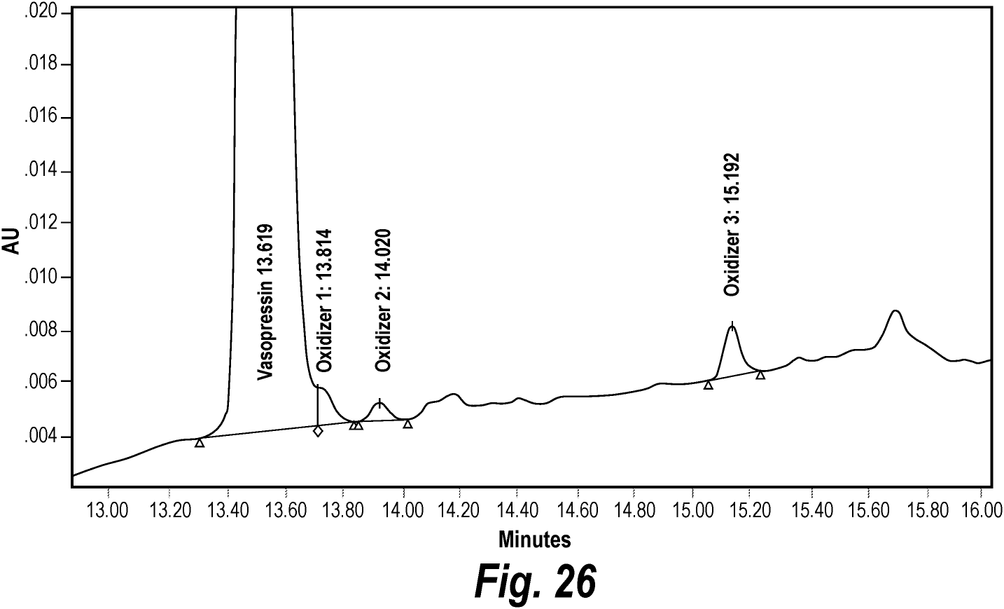

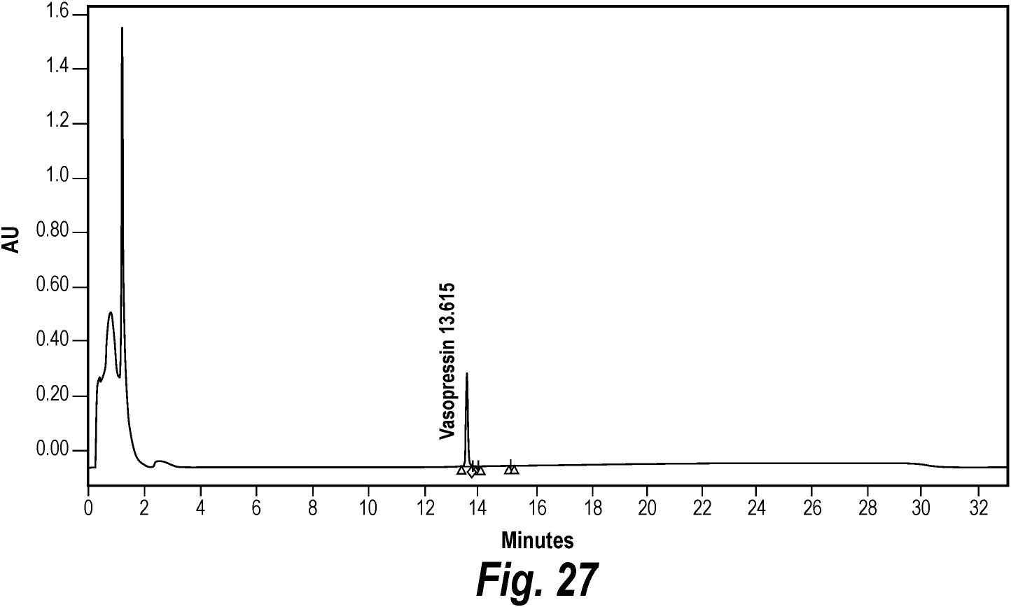

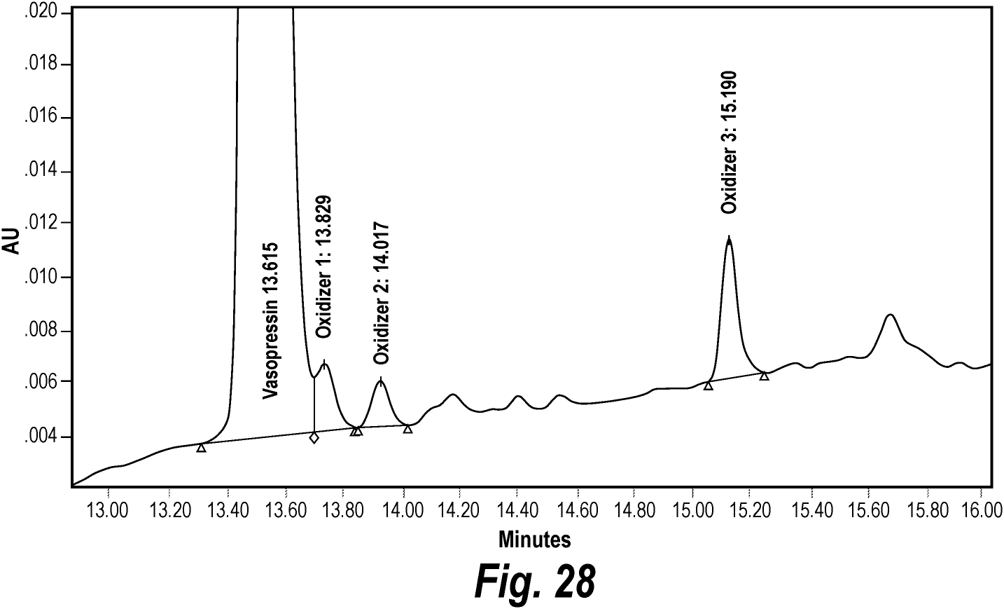

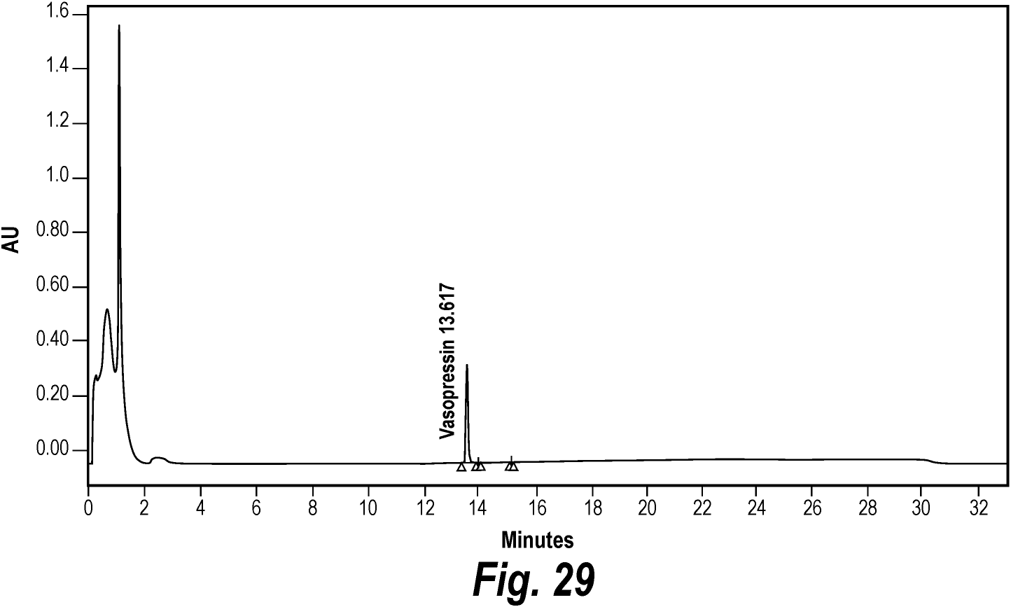

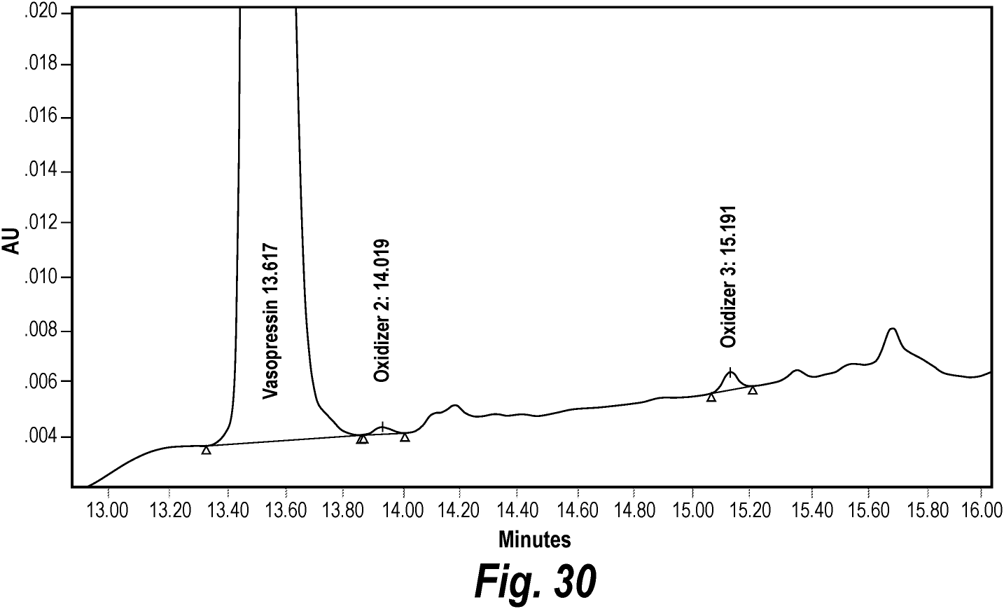

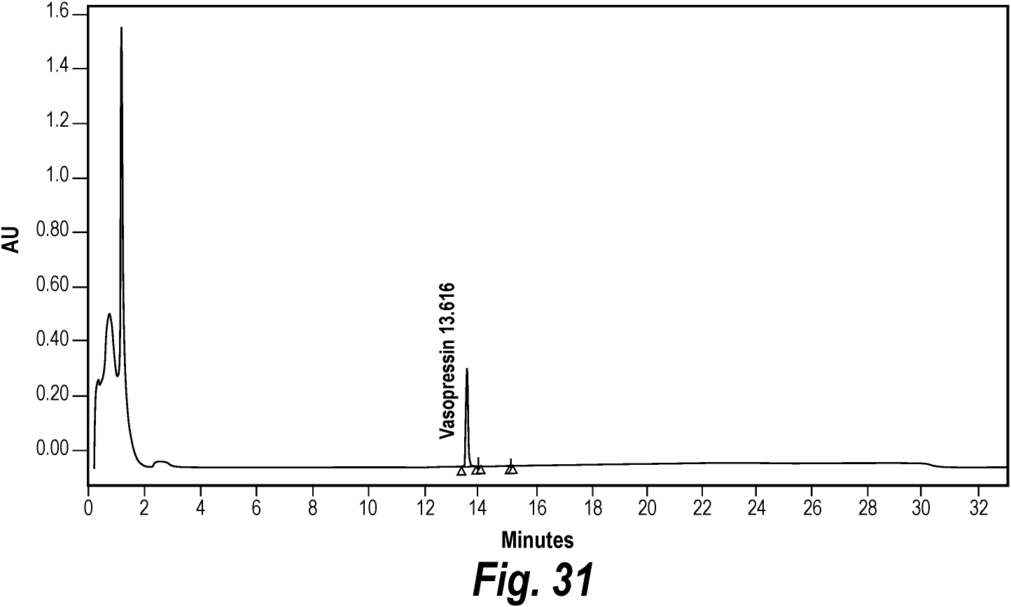

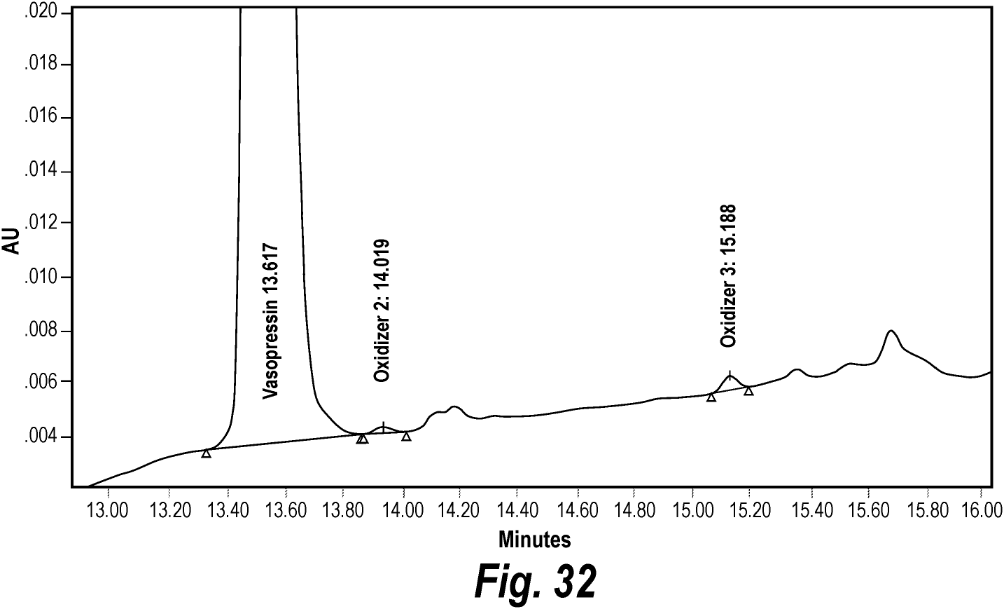

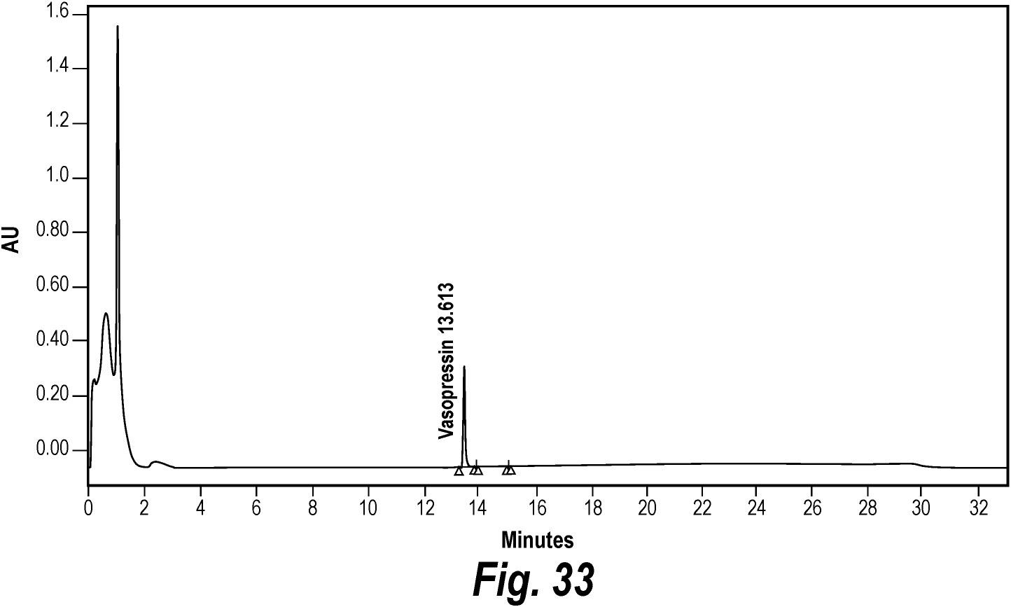

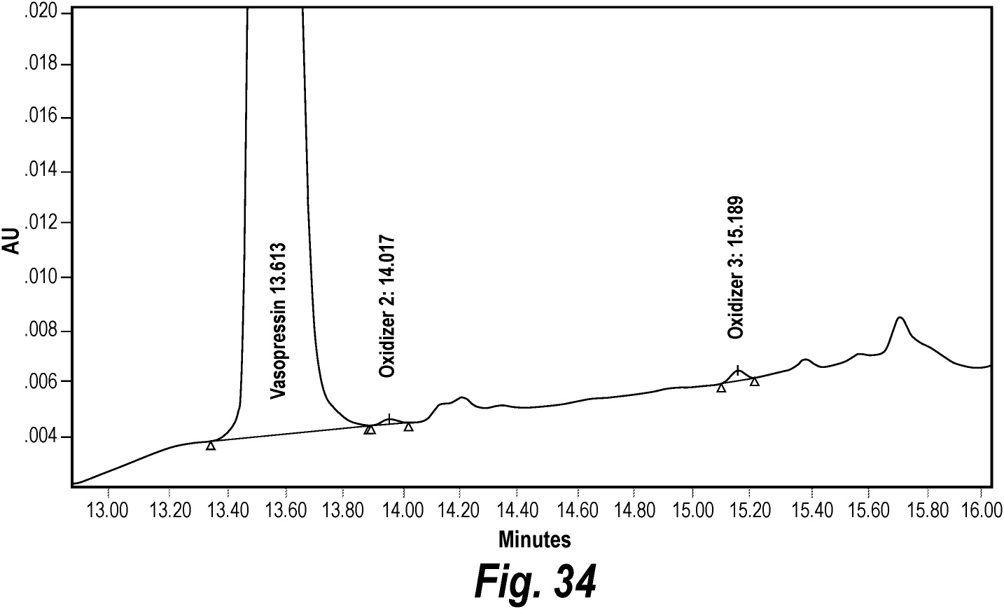

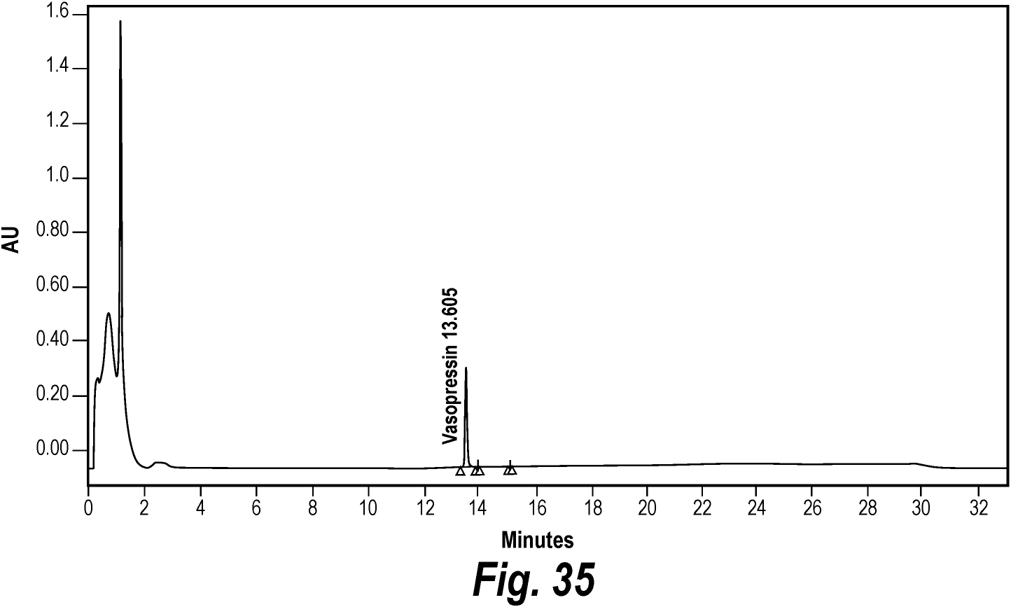

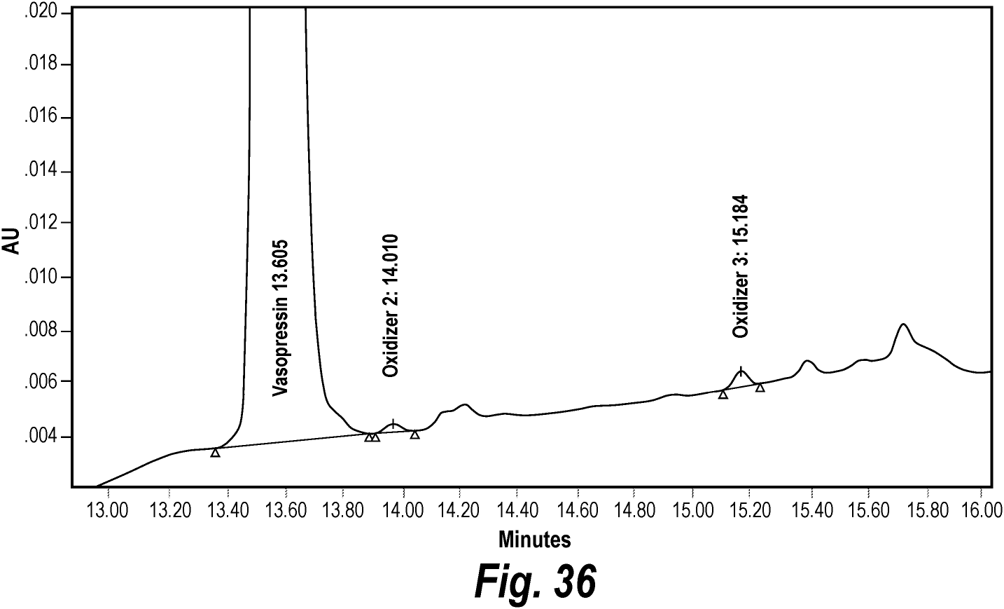

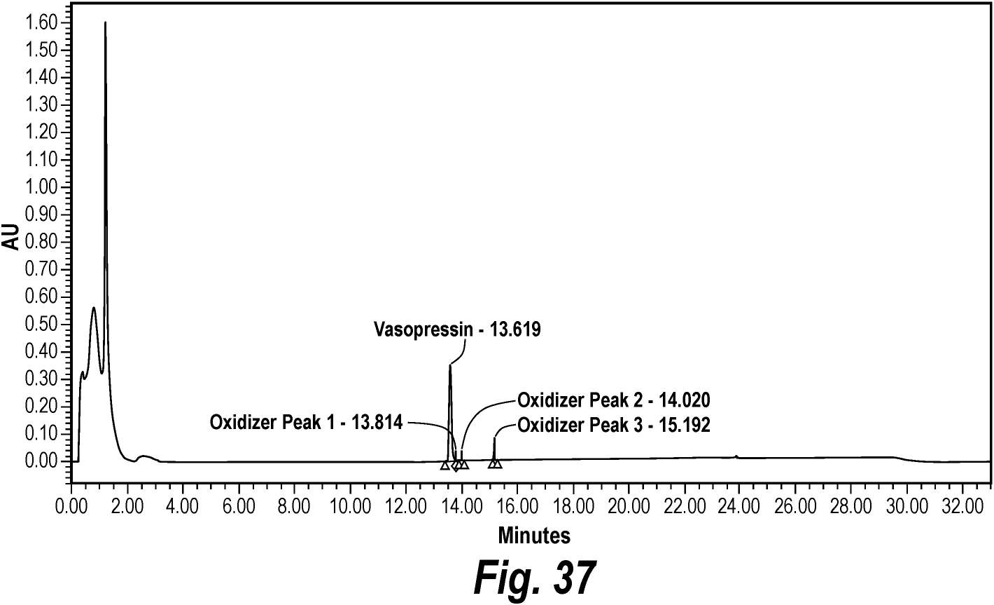

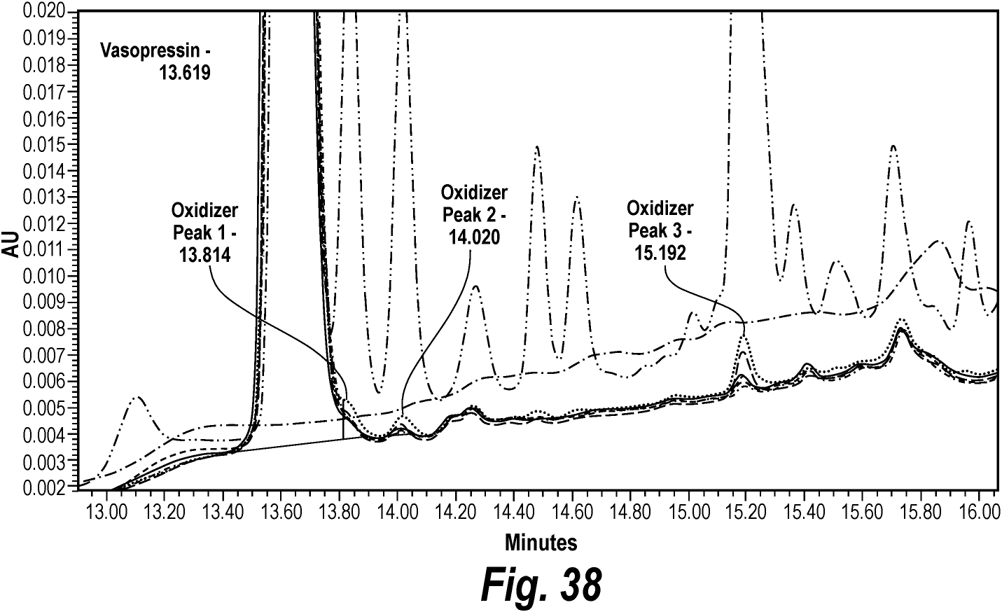

FIGS. 19-38 show the results of conducting HPLC analysis on control and swab samples containing 1000 ng/25 cm², 500 ng/25 cm², 50 ng/25 cm², 25 ng/25 cm², 5 ng/25 cm², 2.5 ng/25 cm², 0.5 ng/25 cm², and 0.25 ng/25 cm² available chlorine. FIGS. 22-38 show impurity peaks of vasopressin at about 13.8, 14.0 and 15.2 minutes. FIG. 21 shows that at the concentration tested (1000 ng/25 cm²) the available chlorine degraded all of the vasopressin as shown by the chromatogram appearing similar to a blank injection. These figures further illustrate the method of the present disclosure may detect trace or residual amounts of oxidizing substances in samples from as low as 0.25 ng/25 cm² or 0.05 ng/mL.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

Any numerical range recited herein includes all values and ranges from the lower value to the upper value. For example, if a concentration range is stated as 1% to 50%, it is intended that values such as 2% to 40%, 10% to 30%, 1% to 3%, or 2%, 25%, 39% and the like, are expressly enumerated in this specification. These are only examples of what is specifically intended, and all possible combinations of numerical values and ranges between and including the lowest value and the highest value enumerated are to be considered to be expressly stated in this application.

The present disclosure may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

What is claimed is:

1. An analytical method for detecting the presence of oxidizing substances by peptide degradation, the method comprising:
mixing a peptide with a sample to create a test preparation; and
detecting impurities of the peptide in the test preparation, wherein detected impurities indicate the presence of oxidizing substances in the sample.

2. The method of claim 1, further comprising identifying the detected impurities of the peptide in the test preparation.

3. The method of claim 1, further comprising measuring the amount of the detected impurities.

4. The method of claim 1, wherein the peptide comprises vasopressin.

5. The method of claim 1, wherein the sample comprises a rinse solution.

6. The method of claim 1, wherein the sample comprises a swab sample.

7. The method of claim 1, wherein the oxidizing substances comprise a chlorine-containing or bromine-containing compound.

8. The method of claim 7, wherein the chlorine-containing compound is selected from the group consisting of sodium hypochlorite, calcium hypochlorite, and sodium dichloroisocyanurate.

9. The method of claim 7, wherein the bromine-containing compound is 1-bromo-3-chloro-5,5-dimethylhydantoin (BCDMH).

10. The method of claim 1, wherein detecting impurities of the peptide in the test preparation comprises analyzing the test preparation using high performance liquid chromatography or ultra-high performance liquid chromatography.

11. The method of claim 1, further comprising collecting the sample from equipment previously exposed to a cleaning solution.

12. The method of claim 11, wherein the cleaning solution comprises a chlorine-containing or bromine-containing compound.

13. The method of claim 1, wherein presence of oxidizing substances in the sample causes degradation of the peptide.

14. An analytical method for detecting the presence of oxidizing substances by peptide degradation, the method comprising:
preparing a test preparation comprising a peptide and a sample, wherein the peptide degrades in the presence of oxidizing substances; and
detecting impurities of the peptide in the test preparation, wherein detected impurities indicate the presence of oxidizing substances in the sample.

15. The method of claim 14, further comprising identifying the detected impurities of the peptide in the test preparation and measuring the amount of each detected impurity.

16. The method of claim 14, wherein the oxidizing substances comprise trace or residual amounts.

17. The method of claim 14, wherein the peptide comprises vasopressin.

18. The method of claim 14, wherein the sample comprises a rinse solution or a swab sample.

19. The method of claim 14, wherein the oxidizing substances comprise a chlorine-containing or bromine-containing compound.

20. The method of claim 14, wherein the detecting impurities of the peptide in the test preparation comprises analyzing the test preparation using high performance liquid chromatography or ultra-high performance liquid chromatography.

21. The method of claim 1, wherein the amount of oxidizing substances present in the sample is from about 0.05 ng/mL to about 100 ng/mL.

22. The method of claim 1, wherein the amount of oxidizing substances present in the sample is from about 0.125 ng/mL to about 50 ng/mL.

23. The method of claim 1, wherein the amount of oxidizing substances present in the sample is from about 0.5 to about 25 ng/mL.
